(19) United States
(12) Patent Application Publication
(10) Pub. No.: US 2013/0172306 A1
(43) Pub. Date: Jul. 4, 2013

(54) USE OF AN ESTROGEN DERIVATIVE FOR THE MANUFACTURE OF PHARMACEUTICAL COMPOSITIONS USEFUL FOR THE TREATMENT AND/OR PREVENTION OF PSYCHIATRIC DISEASES AND FOR THE TREATMENT AND PREVENTION OF SAID DISEASES

(71) Applicants: Tibor Hajszan, Szeged (HU); Laszlo Puskas, Szeged (HU); Bela Ozsvari, Szeged (HU); Lajos Istvan Nagy, Kiskunfelegyhaza (HU)

(72) Inventors: Tibor Hajszan, Szeged (HU); Laszlo Puskas, Szeged (HU); Bela Ozsvari, Szeged (HU); Lajos Istvan Nagy, Kiskunfelegyhaza (HU)

(21) Appl. No.: 13/806,500

(22) PCT Filed: Jun. 28, 2011
(86) PCT No.: PCT/HU11/00060

§ 371 Date: Mar. 12, 2013

(30) Foreign Application Priority Data
Jun. 22, 2010 (HU) P1000335

(51) Int. Cl. A61K 31/565

(52) U.S. Cl. CPC A61K 31/565
USPC 514/182

(57) ABSTRACT

The object of the invention is the use of 17-alpha-estradiol of formula (I) and its derivatives for the treatment and/or prevention of psychiatric clinical pictures. According to the invention, 17-alpha-estradiol and its derivatives can be used preferably as antidepressant drugs, especially preferably as quick-acting antidepressant drugs. According to the invention, 17-alpha-estradiol of formula (I) and its derivatives can also be used for the treatment and/or prevention of postpartum depression, post-menopausal depression, anxiety, schizophrenia, memory disturbance associated with depression or bipolar depression.

The object of our invention is the use of 17-alpha-estradiol for the treatment and/or prevention of psychiatric clinical pictures. Our invention especially relates to the use of 17-alpha-estradiol for the treatment and/or prevention of depression and postpartum depression, post-menopausal depression, anxiety, schizophrenia, memory disturbances associated with depression and for the treatment and/or prevention of bipolar depression. It is a further object of our invention to provide a procedure for preparation of pharmaceutical compositions used for the treatment and/or prevention of the above psychiatric clinical pictures, and such pharmaceutical composition.

17-alpha-estradiol corresponds to the formula (I).

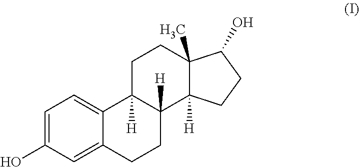

IUPAC name of the compound: estra-1,3,5(10)-triene-3,17α-diol. Other accepted names are: 1,3,5(10)-estratriene-3,17α-diol, 3,17α-dihydroxy-1,3,5(10)-estratriene or epiestradiol.

It is known that 17-alpha-estradiol mitigates DHT induction of PSA gene expression and reduces cellular proliferation. 17-alpha-estradiol inhibits proliferation of LAPC-4 prostate tumour cells in cell cultures and the increase of the tumour in xenograft animals [Kuiao Y et al.: The prostate 67:1719-1728 (2007)]. Neuoroprotective effect of 17-alpha-estradiol was also described in the literature [Dykeus J A: Development of 17-α-estradiol as a neuroprotective therapeutic agent. Ann NY Acad Sci 1052:116-135 (2005)], specifically it was described that 17-alpha-estradiol had a neuroprotective effect on SK—N—SH cells [Green P S et al: J Neurosci 17(2):511-515 (1997)]. The favourable effect of 17-alpha-estradiol in the treatment of Alzheimer's disease was also described. [Onio H et al: Estrogen and non-feminizing estrogen for Alzheimer-disease. Endocrin Journal 50(4):361-367 (2003)].

A procedure for the treatment of estrogen deficiency of the central nervous system was described in the U.S. Pat. No. 6,245,756 during which the patient in need of treatment obtained an efficient quantity of a steroid compound having selective neurotropic transcription action in contrast with the systemic action characteristic of the estrogen compounds.

The aim of our invention is the use of 17-alpha-estradiol in indications different from those known.

The above aim is reached by means of the present second indication invention.

The object of our invention is a procedure for preparation of pharmaceutical compositions in such a way that 17-alpha-estradiol with the formula (I) prepared in a known way

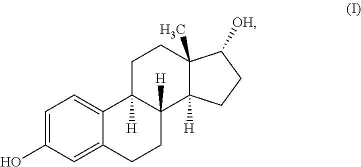

and/or one or more derivatives of 17-alpha-estradiol modified on its alcoholic or phenolic hydroxyl group is/are mixed with inert pharmaceutical carriers and/or excipients and formed to a pharmaceutical composition suitable for the treatment and/or prevention of psychiatric clinical pictures.

In accordance with a preferred embodiment of our invention, pharmaceutical compositions with an antidepressant effect, especially preferably, quick-acting pharmaceutical compositions with an anti-depressant effect are prepared.

In accordance with a specific embodiment of the above indication, pharmaceutical compositions suitable for the treatment and/or prevention of postpartum depression or post-menopausal depression are prepared.

In accordance with a further embodiment of the invention, pharmaceutical compositions suitable for the treatment and/or prevention of anxiety, schizophrenia, memory disturbances associated with depression or bipolar depression are prepared.

A further object of our invention is the use of 17-alpha-estradiol of formula (I) and one or more derivatives thereof

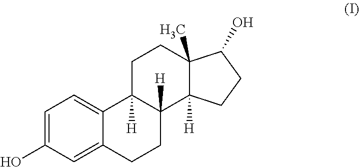

for the treatment and/or prevention of psychiatric clinical pictures.

A preferred embodiment of the above aspect of our invention is the use of 17-alpha-estradiol of formula (I) and its derivatives as an antidepressant agent, especially preferably, a quick-acting antidepressant agent.

A special embodiment of the above use is the use of 17-alpha-estradiol of formula (I) and/or one or more of its derivatives for the treatment and/or prevention of postpartum depression or post-menopausal depression.

In accordance with the above aspect of the invention, 17-alpha-estradiol of formula (I) and its derivatives can also be used for the treatment and/or prevention of anxiety, schizophrenia, memory disturbances associated with depression or bipolar depression.

A further object of our invention is a pharmaceutical composition useful for the treatment and/or prevention of psychiatric clinical pictures, which contains 17-alpha-estradiol of formula (I) and/or one or more of its derivatives as the active agent and inert, solid or liquid, pharmaceutically acceptable carriers and/or excipients.

According to a preferable aspect of our invention, the pharmaceutical composition has an antidepressant effect. According to another preferable aspect the pharmaceutical composition according to the invention has a quick antidepressant effect.

The above aspect of the invention further pertains to postpartum depressive or post-menopausal depressive pharmaceutical compositions.

The pharmaceutical compositions according to the invention are also especially useful for the treatment and/or prevention of anxiety, schizophrenia, memory disturbances associated with depression or bipolar depression.

A further object of our invention is a procedure for the treatment of psychiatric clinical pictures in such a way that a pharmaceutical composition containing a pharmaceutically suitable amount of 17-alpha-estradiol of formula (I) and/or one or more of its derivatives is administered to the patient in need of treatment.

The daily dose of 17-alpha-estradiol of formula (I) is 0.2-300 μg/kg, preferably 0.5-50 μg/kg, especially preferably 3-15 μg/kg.

### A BRIEF DESCRIPTION OF THE FIGURE

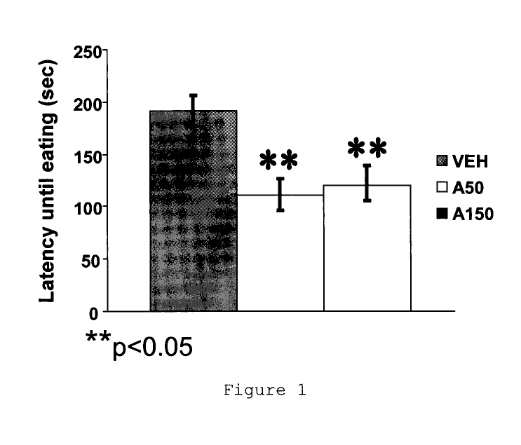

the result of the experiment on the examination of the effect of 17-alpha-estradiol in the test “intake of food inhibited by novel environment” can be seen in FIG. 1.

The term “derivatives of 17-alpha-estradiol” in the context of the present invention means the derivatives modified on the alcoholic hydroxyl group in position 17 and/or the phenolic hydroxyl group in position 3. The derivatives are prepared by esterification of the given hydroxyl group. More exactly, esterification of 17-alpha-estradiol can be performed for example with bivalent acids (e.g. malonic acid, succinic acid, glutaric acid, fumaric acid, maleic acid, etc.) to obtain half esters or with multivalent inorganic acids (e.g. sulphuric acid, phosphoric acid) to obtain esters. According to the invention, the derivatives of 17-alpha-estradiol may be pharmaceutically acceptable salts as well. A few examples of the derivatives of 17-alpha-estradiol are shown below:

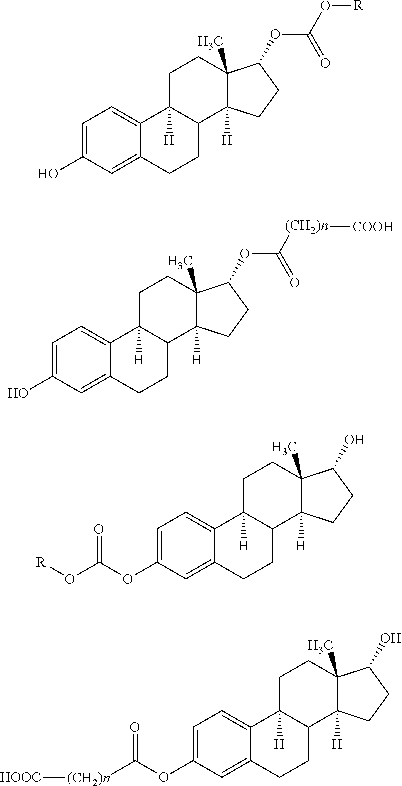

In these structures R may represent a C₁₋₄ alkyl, C₂₋₄ hydroxyalkyl, or a C₂₋₄ aminoalkyl group substituted with an alkyl, cycloalkyl, aryl, or heteroaryl group.

Bio-availability and/or absorption properties of such derivatives are usually better than the corresponding properties of 17-alpha-estradiol, so such compounds may have more favourable pharmacological properties. Such derivatives probably act as pre-drugs of 17-alpha-estradiol. The derivatives are probably more favourable active substances than 17-alpha-estradiol since their solubility in water is improved by derivatization so, for example, those are absorbed better from the stomach. So the derivatives having better solubility in water than that of 17 alpha estradiol can be considered as preferred derivatives from the aspect of the invention. The functional groups and molecule parts which can be used on the molecule skeleton for improvement of the solubility in water are obvious for a person skilled in the pharmaceutical research.

For achieving solubility in water, solubilising agents, e.g. cyclodextrins can also be used. For example, in relation to poorly soluble prednisolone, beta cyclodextrine was described as a solubilising agent where prednisolone was completely absorbed in a regulated way from tablets containing hydroxypropyl methyl cellulose as a matrix. [Rao V M, Haslam J L, Stella V J: J Pharm Sci. 2001 July; 90(7):807-816.].

It was not predictable that 17-alpha-estradiol of formula (I) had an anti-depressant effect. It is known that depression occurs twice as often in women than in men [Kessler R C at al.: Sex and depression in the National Comorbidity Survey. I: Lifetime prevalence, chronicity and recurrence. J Affect Disord 29:85-96 (1993)]; many people explain this difference with the presence of 17-beta-estradiol in women [Steiner M at al.: Hormones and mood: from menarche to menopause and beyond. J Affect Disord 74:67-83 (2003)]. Accordingly, it is supported by many preclinical and clinical studies that 17-beta-estradiol has no antidepressant effect. [Morrison M F et al.: Lack of efficacy of estradiol for depression in postmenopausal women: a randomized, controlled trial. Biol Psychiatry 55:406-412 (2004); Resnick S M et al.: Effects of combination estrogen plus progestin hormone treatment on cognition and affect. J Clin Endocrinol Metab 91:1802-1810 (2006); LaPlant Q et al.: Role of nuclear factor kappaB in ovarian hormone-mediated stress hypersensitivity in female mice. Biol Psychiatry 65:874-880 (2009); Autry A E et al.: Gender-specific impact of brain-derived neurotrophic factor signaling on stress-induced depression-like behavior. Biol Psychiatry 66:84-90 (2009)]. According to the invention, 17-alpha-estradiol and its derivatives can be used preferably as antidepressants, especially preferably as quick-acting antidepressants. The advantage of the latter use is the very quick effect of the composition. According to the state of art, the known antidepressants have to be taken for 3-4 weeks to achieve a favourable effect, while the pharmaceutical composition according to the invention achieves the favourable effect in few hours. Therefore, 17-alpha-estradiol is also useful for ambulant treatment in the indication according to the invention (e.g. after a suicide attempt).

The pharmaceutical composition according to the invention contains the active agent in an amount of 0.1-95 w/w %, preferably 5-50 w/w %, expediently 1-10 w/w %.

The pharmaceutical composition is preferably suitable for oral, parenteral, subcutaneous or rectal administration.

The orally administrable solid pharmaceutical compositions include powders, capsules, tablets, film-coated tablets, microcapsules, etc. and may contain binding agents, e.g. gelatine, sorbitol, polyvinyl pyrrolidone, etc.; filling agents e.g. lactose, glucose, starch, calcium phosphate, etc; tabletting aids, e.g. magnesium stearate, talc, polyethylene glycol, silicon dioxide, etc.; wetting agents e.g. sodium lauryl sulphate, etc. as carriers.

The orally administrable liquid pharmaceutical compositions include solutions, suspensions or emulsions containing a suspending agent, e.g. gelatine, carboxy methyl cellulose, etc., an emulsifier, e.g. sorbitan monooleate, etc.; a solvent, e.g. water, oils, glycerol, propylene glycol, ethanol; preservatives, e.g. p-hydroxy-benzoic acid methyl or propyl ester, etc. as carriers.

Parenterally administrable pharmaceutical compositions usually consist of a sterile solution of the active substance.

The examples mentioned above and other dosage forms are known themselves according to the state of art, see e.g. the handbook Remington's Pharmaceutical Sciences, 18th edition, Mack Publishing Co., Easton, USA (1990).

The pharmaceutical composition usually contains a unit dose. The characteristic daily dose for an adult is 0.2-300 μg active agent per 1 kg body weight, preferably 0.5-50 μg/kg body weight, especially preferably 3-15 μg/kg. The daily dose can be administered in one ore more portions. The actual dose depends on several factors and it will be determined by the physician.

The pharmaceutical composition is prepared by mixing the active agent with one or more carriers and forming a pharmaceutical composition from the mixture in a way known itself. The suitable methods are known from the art, for example the above mentioned handbook of Remington's Pharmaceutical Sciences.

The further details of our invention are described in the following example without limiting the invention to the example.

### EXAMPLE

### Quick Antidepressant and Anti-Anxiety Effect of 17-Alpha-Estradiol

In the course of our experiment the active agent 17-alpha-estradiol of formula (I) was tested in the animal model “novelty suppressed feeding” (NSF). NFS is a conflict test during which the subject of the test has to resolve a conflict between the stimulus of urgent food intake and the anxiety induced by a novel environment [Bodnoff S R et al.: The effects of chronic antidepressant treatment in an animal model of anxiety. Psychopharmacology (Berl) 95:298-302 (1988)]; Bodnoff S R et al.: A comparison of the effects of diazepam versus several typical and atypical anti-depressant drugs in an animal model of anxiety. Psychopharmacology (Berl) 97:277-279 (1989)]. The NSF model has widespread use in studying efficiency and mechanism of antidepressant drugs and in identification of new antidepressant drugs [Santarelli L et al.: Requirement of hippocampal neurogenesis for the behavioral effects of antidepressants. Science 301:805-809 (2003); Warner-Schmidt J L et al.: VEGF is an essential mediator of the neurogenic and behavioral actions of antidepressants. Proc Natl Acad Sci USA 104:4647-4652 (2007); Banasr M et al.: Glial loss in the prefrontal cortex is sufficient to induce depressive-like behaviors. Biol Psychiatry 64:863-870 (2008); Greene J et al.: Vascular endothelial growth factor signaling is required for the behavioral actions of antidepressant treatment: pharmacological and cellular characterization. Neuropsychopharmacology 34:2459-2468 (2009)]. Thus, for example, the key role of neurogenesis in the positive behavourial response on treatment with an antidepressant was also proved by NSF [Santarelli L et al.: Requirement of hippocampal neurogenesis for the behavioral effects of antidepressants. Science 301:805-809 (2003)]. A key property of NSF is that it models the delayed effect of antidepressant drugs excellently [Bodnoff S R et al.: The effects of chronic antidepressant treatment in an animal model of anxiety. Psychopharmacology (Berl) 95:298-302 (1988)], thus enabling screening of quick-acting antidepressants.

Female Sprague-Dawley rats (175-200 g; Charles River Laboratories, Hungary) were used in our experiment. In the first step, the ovaries of the animals were removed under chloral hydrate general anesthesia (in 400 mg/kg physiological salt solution, i.p.) to eliminate the cyclic effect of the changes in the estrogen level on the behaviour of the animals [Hajszan T et al.: Effects of estradiol on learned helplessness and associated remodelling of hippocampal spine synapses in female rats. Biol Psychiatry 67:168-174 (2010)]. After a recovery of two weeks the animals were distributed in 3 treatment groups (12 rats/group), and after food deprivation of 24 hours, the groups obtained 0, 50, and 150 mg/kg active agent 17-alpha-estradiol in 0.5 ml of solvent [(1% ethanol (Sigma, Hungary), 1% DMSO (Sigma), 15% Solutol (BASF, Germany), 83% physiological salt solution)]. At first, 17-alpha-estradiol was dissolved in a 1:1 mixture of ethanol and DMSO in a concentration of 15 mg/ml, then a solution containing the active substance was made from this solution by dilution, the solution obtained was transferred directly into the stomach of the animals through a stomach tube. Two hours after the administration of the active agent the animals were tested in the NSF model.

At the beginning of the NSF test the test animal was placed in a corner of a 75×75×50 cm dark Plexiglas cage. In the middle of the cage, well illuminated, three standard rat food sticks (Akronom Kft., Hungary) were placed. Apart from the food, the cage contained no object or mark. Maximum six minutes were allowed for the rat to scout the cage and to start to eat from the food placed in the middle of the cage. The time from the start of the test to the beginning of food intake (first biting) was measured and recorded. Every test was recorded by means of a video camera suspended over the cage. The floor and all the four walls of the cage were thoroughly cleaned using 70% alcohol and the food was replaced with fresh food after each test. The tests were carried out between 10:00-16:00 hours in a semi dark room where only the cage was illuminated with medium intensity white light.

After the NSF test the animals were replaced to their original cage where they got food and their food intake was measured for further 5 minutes to check whether the given treatment had altered the appetite of the animals. The configuration of the NSF test used by us is the same as the method published earlier several times [Warner-Schmidt J L et al.: VEGF is an essential mediator of the neurogenic and behavioral actions of antidepressants. Proc Natl Acad Sci USA 104:4647-4652 (2007); Banasr M et al.: Glial loss in the prefrontal cortex is sufficient to induce depressive-like behaviors. Biol Psychiatry 64:863-870 (2008); Greene J et al.: Vascular endothelial growth factor signaling is required for the behavioral actions of antidepressant treatment: pharmacological and cellular characterization. Neuropsychopharmacology 34:2459-2468 (2009)].

The group mean and the standard error of the mean (SEM) were calculated from the results. At first a one-way ANOVA analysis of the data was carried out then the treatment groups were compared in pairs using the Tukey post-hoc test. p<0.05 was accepted as the criterion of significance. The statistical tests were run with the software package SPSS (Chicago, Ill., USA). Our results were plotted as group mean±standard error of the mean (SEM).

### MEANING OF THE ABBREVIATIONS IN FIG. 1

meaning of the asterisks: p<0.05;
VEH=control, group of vehiculum (carrier with no active agent);
A50=animals treated with an solution containing 50 μg/kg 17-alpha-estradiol active agent; and
A150=animals treated with a solution containing 150 μg/kg 17-alpha-estradiol active agent.

From the diagram in FIG. 1 it can be seen that the ovariectomised rats placed into a novel environment, which had obtained 17-alpha-estradiol, started food intake in a shorter time than the control animals treated only with the carrier. The difference was significant in the case of both treated groups. The shorter time means that the behaviour corresponding to depression (inhibition of food intake) was reduced in the rats treated, which is a clear proof of the antidepressant effect.

1: A procedure for preparation of pharmaceutical compositions, characterized in that 17-alpha-estradiol, with the formula (I) 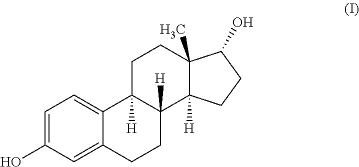
prepared in the known way, and/or one or more derivatives of 17-alpha-estradiol modified on its alcoholic or phenolic hydroxyl group is/are mixed with inert pharmaceutical carriers and/or excipients and formed to a pharmaceutical composition useful for the treatment and/or prevention of psychiatric clinical pictures.

2: A procedure according to claim 1, characterized in that pharmaceutical compositions with an antidepressant effect are prepared.

3: A procedure according to claim 2, characterized in that quick-acting antidepressant compositions are prepared.

4: A procedure according to claim 2, characterized in that pharmaceutical compositions useful for the treatment of postpartum depression and/or post-menopausal depression are prepared.

5: A procedure according to claim 1, characterized in that pharmaceutical compositions useful against anxiety, for the treatment of schizophrenia, memory disturbances associated with depression or bipolar depression are prepared.

6: Use of 17-alpha-estradiol of formula (I) and/or one or more derivatives of 17-alpha-estradiol modified on its alcoholic and/or phenolic hydroxyl group for the treatment and/or prevention of psychiatric clinical pictures.

7: Use according to claim 6 for the treatment of depression.

8: Use according to claim 7 as a quick-acting antidepressant.

9: Use according to claim 7 for the treatment of postpartum depression or post-menopausal depression.

10: Use according to claim 6 for the treatment of memory disturbances associated with depression, anxiety, schizophrenia or bipolar depression.

11: A pharmaceutical composition useful for the treatment and/or prevention of psychiatric clinical pictures containing 17-alpha-estradiol of formula (I) and/or one or more derivatives of 17-alpha-estradiol modified on its alcoholic or phenolic hydroxyl group as the active agent and inert, solid or liquid, pharmaceutically suitable carriers and/or excipients.

12: A pharmaceutical composition according to claim 11 with an antidepressant effect.

13: A pharmaceutical composition according to claim 12 with a quick-acting antidepressant effect.

14: A pharmaceutical composition according to claim 12 with postpartum antidepressant or post-menopausal antidepressant effect.

15: A pharmaceutical composition according to claim 11 useful for the treatment and/or prevention of anxiety, schizophrenia, memory disturbances associated with depression or bipolar depression.

16: A procedure for the treatment and/or prevention of psychiatric clinical pictures, especially depression, characterized in that a pharmaceutically efficient quantity of a pharmaceutical composition containing 17-alpha-estradiol of formula (I) and/or one or more derivatives of 17-alpha-estradiol modified on its alcoholic and/or phenolic hydroxyl group is administered to the patient in need of treatment.

17: A procedure according to claim 16, characterized in that 17-alpha-estradiol of formula (I) or its derivative is used in a quantity of 0.2-300 μg/kg, preferably 0.5-50 μg/kg, especially preferably 3-15 μg/kg.

18: A procedure according to claim 3, characterized in that pharmaceutical compositions useful for the treatment of postpartum depression and/or post-menopausal depression are prepared.

19: A pharmaceutical composition according to claim 13 containing 17-alpha-estradiol in an amount sufficient to elicit postpartum antidepressant or post-menopausal antidepressant effect.

20: The pharmaceutical composition of claim 11 in liquid form and comprising about 0.1 to about 95 weight percent 17-alpha-estradiol and a pharmaceutically suitable liquid carrier.
